(19) United States
(12) Patent Application Publication
(10) Pub. No.: US 2025/0281513 A1
(43) Pub. Date: Sep. 11, 2025

(54) USE OF PSYCHEDELICS TO TREAT DEMENTIA

(71) Applicants: MYND LIFE SCIENCES INC., Kelowna (CA); The University of British Columbia, Vancover (CA)

(72) Inventor: Wilfred A. JEFFERIES, Surrey (CA)

(73) Assignees: MYND LIFE SCIENCES INC., Kelowna, BC (CA); The University of British Columbia, Vancouver, BC (CA)

(21) Appl. No.: 18/285,019

(22) PCT Filed: Mar. 29, 2022
(86) PCT No.: PCT/CA2022/050468

§ 371 Date: Sep. 29, 2023

Related U.S. Application Data
(60) Provisional application No. 63/167,897, filed on Mar. 30, 2021.

(51) Int. Cl. A61K 31/675; A61K 31/4045; A61P 25/28

(52) U.S. Cl. CPC A61K 31/675; A61K 31/4045; A61P 25/28

(57) ABSTRACT

The present invention provides use of psychedelics to prevent and/or treat dementia. In certain embodiments, the one or more psychedelics inhibit neuroinflammation, inhibit cerebral angiogenesis, promote neuroplasticity, down regulate APP and/or Tau or a combination thereof and thereby treat, delay and/or prevent dementia.

### REFERENCE TO SEQUENCE LISTING SUBMITTED ELECTRONICALLY

The content of the electronically submitted sequence listing (Name:2024-04-25 Seq Listing_ST25 (4753-105US).txt; Size: 27530 bytes; and Date of Creation: Apr. 24, 2024) filed in this application is herein incorporated by reference in its entirety.

### FIELD OF THE INVENTION

The present invention relates to the field of therapeutics, in particular as it relates to the use of psychedelics to treat dementia.

### BACKGROUND OF THE INVENTION

Alzheimer's disease, a progressive neurodegenerative disorder, causes loss of memory and other intellectual abilities leading to dementia. The exact cause of this disease is still unknown, and the mechanism of pathogenesis is highly debated. Amyloid beta (Aβ) peptide is central to the disease along with the cerebrovascular dysfunction and impaired cerebral blood flow (CBF).

In addition, studies have shown that neuroinflammation plays a fundamental role in the progression of the neuropathological changes that are observed in Alzheimer's disease. The neuroinflammation observed in Alzheimer's disease is not only associated with neurodegeneration but it also facilitates amyloid β plaques and neurofibrillary tangles pathologies. In addition, neuroinflammation in Alzheimer's disease leads to up-regulation of mediators that initiate pathological angiogenesis.

Neuroinflammation is also associated with the pathogenesis of other dementias including but not limited to Parkinson's disease dementia, frontotemporal dementia, vascular dementia and Lewy body dementia.

Inflammation and immune responses are tightly controlled cellular mechanisms that help maintain cellular homeostasis. These mechanisms are governed by several proteins that regulate a cascade of downstream effectors.

ABCF1 is a strong negative regulator of pro-inflammatory responses and changes in ABCF1 activity/expression is associated with a number of inflammatory and/or autoimmune diseases. ABCF1 mediates M2 polarization. The M1 phenotype is stimulated by microbial products or pro-inflammatory cytokines [IFN-γ, TNF, or Toll-like receptor (TLR) ligands]. M1 macrophages produce pro-inflammatory cytokines including but not limited to TNFα, IL-1, IL-6, IL-12, Type I IFN, CXCL1-3, CXCL-5, and CXCL8-10. M2 macrophages resolve inflammation, help tissue healing, tolerate self-antigens and certain neoantigens. M2 macrophages produce anti-inflammatory cytokines such as IL-10.

Microglia are the resident macrophages of the CNS. It has been proposed that microglia in the aging brain mainly present as the M1 proinflammatory phenotype which results in increased neuroinflammation and leads to neurotoxicity to CNS cells.

ABCF1 expression is higher in APP/PS1 mice as compared to Wild type mice (Jorda et al. Int. J. Biol. Sci 2019 15(2):453-463). KR101574766B1 teaches a biomarker composition for diagnosing Alzheimer's disease which includes ABCF1 in CSF.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention provides the use of psychedelics to treat dementia. In one aspect of the present invention there is provided a method of inhibiting neuroinflammation by upregulating the expression and/or activity of ABCF1.

In another aspect of the present invention, there is provided a method of inhibiting neuroinflammation and thereby treating, delaying and/or preventing dementia by administering one or more psychedelics.

In another aspect of the present invention, there is provided a method of inhibiting neuroinflammation and thereby treating, delaying and/or preventing dementia by administering psilocybin, psilocybin derivatives and psilocybin like compounds alone or in combination with other therapeutics.

In another aspect of the present invention, there is provided a method of thereby treating, delaying and/or preventing dementia by administering one or more psychedelics alone or in combination with other therapeutics.

In another aspect of the present invention, there is provided a method of treating, delaying and/or preventing dementia by administering psilocybin, psilocybin derivatives and psilocybin like compounds alone or in combination with other therapeutics.

### BRIEF DESCRIPTION OF THE FIGURES

These and other features of the invention will become more apparent in the following detailed description in which reference is made to the appended drawings.

FIG. 1 illustrates that Escitalopram induces ABCF1 expression in a Macrophage cell line FIG. 2 illustrates the effect of psylocibin, psylocin and their analogs on ABCF1 transcription in a macrophage cell line. ES=escitalopram; PSYB=Psylocibin; PSIC=Psilocin; DMT=4-Acetoxy-N, N-dimthyltryptamine; APF=O-Acetyl Psilocin Fumerate, and AOI=4-acetoxyindole.

FIG. 3 illustrates the effect of Amyloid β protein (Aβ1-16) on ABCF1 transcription in a macrophage cell line.

FIG. 4 illustrates the effect of Amyloid β protein (Aβ1-16) on ABCF1 transcription in a macrophage cell line.

FIG. 5 illustrates ABCF1 expression in RAW cells treated with Psilosybin.

FIG. 6 illustrates expression levels of Hif-α and ABCF1 2 hours post 500 nM Psilosybin treatment.

FIG. 7 illustrates ABCF1 expression in RAW cells treated for 2 hours, 24 hours and 26 hours.

FIG. 8 illustrates real time quantitative PCR showing expression levels of Hif-α and ABCF1 in RAW cells post Aβ 1-16 treatment for 2 hours.

FIG. 9 illustrates down regulation of amyloid precursor protein (APP) in RAW cells post 2 hours treatment with psychedelic drugs (APF=O-Acetyl Psilocin Fumerate, and AOI=4-acetoxyindole).

FIG. 10 illustrates down regulation of Tau in RAW cells post 2 hours treatment with psychedelic drugs (DMT=4-Acetoxy-N, N-dimthyltryptamine).

### DETAILED DESCRIPTION OF THE INVENTION

The present invention relates to methods of preventing and/or treating dementia. In certain embodiments, the dementia is Alzheimer's Disease. In specific embodiments, the Alzheimer's disease is early onset familial or late onset sporadic. In certain embodiments, the other dementias are selected from Downs syndrome dementia, vascular dementia, frontal temporal, Pick's disease, tauopathies, Progressive Supranuclear Palsy, Corticobasal Degeneration, Parkinson's disease, Parkinson's disease dementia, frontotemporal dementia, vascular dementia, Lewy body dementia, mixed dementia, multiple sclerosis, ALS, HD and Stroke.

Neuroinflammation plays a role in the pathogenesis of a number of dementias. Such dementias include but are not limited to Alzheimer's disease (AD), Parkinson's disease dementia (PDD), frontotemporal dementia (FTD), Lewy body dementia (LBD) and mixed dementia. Accordingly, in certain embodiments, there is provided a method of preventing and/or treating dementia by inhibiting neuroinflammation. In specific embodiments, the dementia is selected from the group consisting of Alzheimer's disease (AD), Parkinson's disease dementia (PDD), frontotemporal dementia (FTD), Lewy body dementia (LBD) and mixed dementia. In more specific embodiments, the dementia is Alzheimer's disease.

ABCF1 is a strong negative regulator of pro-inflammatory responses. An increase in activity/expression of ABCF1 may result in a decreased inflammatory response. This decreased inflammatory response may result in decreased neurotoxicity. Accordingly, in certain embodiments, there is provided a method of inhibiting neuroinflammation by upregulating the expression and/or activity of ABCF1. In certain embodiments, inhibiting neuroinflammation prevents and/or treats dementia. Accordingly, in certain embodiments there is provided a method of preventing and/or treating dementia by upregulating the expression and/or activity of ABCF1. In specific embodiments, the dementia is selected from the group consisting of Alzheimer's disease (AD), Parkinson's disease dementia (PDD), frontotemporal dementia (FTD), Lewy body dementia (LBD) and mixed dementia. In more specific embodiments, the dementia is Alzheimer's disease.

Non-limiting examples of methods to enhance expression and/or activity of ABCF1, include administration of ABCF1, administration of a nucleic acid or vector which encodes the ABCF1 or administration of one or more molecules which enhance expression of the polypeptide of ABCF1. Escitalopram is a known enhancer of the ABCF1 pathway. In addition, as demonstrated herewith, a number of psychedelics, including psilocybin, psilocin and their analogues including 4-Acetoxy-N, N-dimthyltryptamine; O-Acetyl Psilocin Fumerate, and 4-acetoxyindole enhance expression of ABCF1.

A number of psychedelics have been shown to have anti-angiogenic properties. Cerebral angiogenesis has also been shown to play a role in the pathogenesis of some dementias, including but not limited to Alzheimer's disease. In addition, to a number of psychedelics having been shown to have anti-inflammatory and/or anti-angiogenic properties, a number of psychedelics have also been shown to promote neuroplasticity (i.e., the ability of the brain to form and reorganize synaptic connections). Promoting the ability of the brain to form and reorganize synaptic connections may delay the progression of dementia.

A number of psychedelics have been shown here to down regulate APP and/or Tau, proteins involved in the pathogenesis of Alzheimer's disease.

Accordingly, in certain embodiments, the present invention provides a method of delaying, preventing and/or treating dementia, including but not limited to Alzheimer's Disease with one or more psychedelics. The one or more psychedelics may delay, prevent and/or treat by inhibiting neuroinflammation, inhibiting cerebral angiogenesis, promoting neuroplasticity, down regulating APP and/or Tau or a combination thereof.

Reducing APP restores the blood brain barrier (BBB), removes plaques and restores cognition in Alzheimer's disease and other diseases. In certain embodiments, the present invention provides methods of restoring the blood brain barrier in Alzheimer's disease and other diseases.

In certain embodiments, the present invention provides methods of restoring cognition in Alzheimer's disease.

In certain embodiments, the present invention provides a method of improving cognitive function in a person having dementia, including but not limited to Alzheimer's Disease, by administering one or more psychedelics alone or in combination with other therapeutics. In specific embodiments, there is provided a method of improving cognitive function in a person having dementia, including but not limited to Alzheimer's Disease, by administering psilocybin, psilocybin derivatives and psilocybin like compounds alone or in combination with other therapeutics. As used herein, “improving cognitive function” means to improve memory, learning capacity, communication language, thinking, decision making, judgment, and/or attention in a subject receiving treatment according to a method of the invention in comparison to the performance of the subject prior to receiving the treatment. The cognitive function can be evaluated using any of a variety of known tests for measuring and monitoring changes in cognitive function.

In certain embodiments, the present invention provides a method of improving movement, walking, balance, speech, swallowing, vision, mood, behavior, and thinking in a person having dementia, including but not limited to Alzheimer's Disease, by administering one or more psychedelics alone or in combination with other therapeutics. In specific embodiments, there is provided a method of improving movement, walking, balance, speech, swallowing, vision, mood, behavior, and thinking in a person having dementia, including but not limited to Alzheimer's Disease, by administering psilocybin, psilocybin derivatives and psilocybin like compounds alone or in combination with other therapeutics.

In certain embodiments, the present invention provides a method of delaying the loss of cognitive function in a person having dementia, including but not limited to Alzheimer's Disease, by administering one or more psychedelics alone or in combination with other therapeutics. In specific embodiments, there is provided a method of delaying the loss of cognitive function in a person having dementia, including but not limited to Alzheimer's Disease, by administering psilocybin, psilocybin derivatives and psilocybin like compounds alone or in combination with other therapeutics. As used herein, “delaying the loss of cognitive function” is meant to delay the loss of memory, learning capacity, communication language, thinking, decision making, judgment, and/or attention in a subject receiving treatment according to a method of the invention in comparison to the average onset of loss of cognitive function observed for untreated subjects at the same stage of disease. The cognitive function can be evaluated using any of a variety of known tests for measuring and monitoring changes in cognitive function.

In certain embodiments, the present invention provides a method of reducing the severity of dementia in a person having dementia, including but not limited to Alzheimer's Disease, by administering one or more psychedelics alone or in combination with other therapeutics. In specific embodiments, there is provided a method of reducing the severity of dementia in a person having dementia, including but not limited to Alzheimer's Disease, by administering psilocybin, psilocybin derivatives and psilocybin like compounds alone or in combination with other therapeutics. As used herein, “reducing the severity of dementia” means to reduce one or more symptoms of dementia in a subject receiving treatment according to a method of the invention in comparison to the performance of the subject prior to receiving the treatment. The symptoms of dementia can be evaluated using any of a variety of tests known in the art.

In certain embodiments, the present invention provides a method of delaying the onset of symptoms of dementia in a person, including but not limited to symptoms of Alzheimer's Disease, by administering one or more psychedelics alone or in combination with other therapeutics. In specific embodiments, there is provided a method of delaying the onset of dementia in a person having dementia, including but not limited to Alzheimer's Disease, by administering psilocybin, psilocybin derivatives and psilocybin like compounds alone or in combination with other therapeutics. As used herein, “delaying the onset of dementia” means to delay one or more symptoms of dementia in a subject receiving treatment according to a method of the invention in comparison to the average onset of dementia observed for untreated subjects at the same stage of disease. The symptoms of dementia can be evaluated using any of a variety of tests known in the art.

Any psychedelic having anti-inflammatory properties, anti-angiogenic properties, neuroplasticity promoting activities or a combination thereof may be used in the methods of the present invention. In specific embodiments, the compound is a psilocybin, psilocybin derivatives and psilocybin like compounds. Exemplary compounds include but are not limited to Psilocybin ([3-(2-Dimethylaminoethyl)-1H-indol-4-yl]dihydrogen phosphate), Psilocybin (zwitterion form), Psilocin (4-hydroxy-N,N-dimethyltryptamine), Serotonin (5-Hydroxytryptamine), DMT (N,N-Dimethyltryptamine), Lysergic acid diethylamide (LSD, (6aR,9R)—N,N-diethyl-7-methyl-4,6,6a,7,8,9-hexahydroindolo[4,3-fg]quinoline-9-carboxamide, psilocin iminoquinone, psilocin o-quinone, Trimethylglycine (TMG), Phenyl hydrogen sulfate and indoxyl sulfate.

In certain embodiments, the molecule is any one of the following listed in the table below:

| Common Name | Formal Name | Natural |
|---|---|---|
| Psilocybin | [3-(2-Dimethylaminoethyl)-1H-indol-4- | YES |
| | yl] dihydrogen phosphate, or | |
| | O-phosphoryl-4-hydroxy-N,N- | |
| | dimethyl-tryptamine | |
| Major Metabolites | | |
| Psilocin | 4-hydroxy-N,N-dimethyltryptamine | YES |
| Psilocin-O-glucuronide | [3-(2-Dimethylaminoethyl)-1H-indol-4- | YES |
| | yl] glucuronide | |
| Psilocin iminoquinone | Psilocin iminoquinone | YES |
| Psilocin o-quinone | Psilocin o-quinone | YES |
| Indoylalkylamines | | |
| Serotonin | 5-Hydroxytryptamine | YES |
| Mescaline | 3,4,5-Trimethoxyphenethylamine | YES |
| DMT | N,N-Dimethyltryptamine | YES |
| 5-methoxy-DMT | 5-methoxy-N,N-Dimethyltryptamine | YES |
| Lysergic acid | (6aR,9R)-N,N-diethyl-7-methyl- | |
| diethylamide (LSD) | 4,6,6a,7,8,9-hexahydroindolo[4,3- | |
| | fg]quinoline-9-carboxamide | |
| Phenylethylamines | | |
| Noradrenaline | 2,5-Dimethoxy-4-methylamphetamine | YES |
| DOM | 2,5-Dimethoxy-4-methylamphetamine | |
| DOI | 2,5-Dimethoxy-4-iodoamphetamine | |
| DOB | 2,5-Dimethoxy-4-bromoamphetamine | |
| DOC | 2,5-Dimethoxy-4-chloroamphetamine | |
| DOF | 2,5-Dimethoxy-4-fluoroamphetamine | |
| Cathinone | benzoylethanamine, or β-keto- | YES |
| | amphetamine | |
| Predicted Compounds | | |
| Other sugar analogs of | Various, replacing the glucuronide | |
| psilocin-O-glucuronide | with an alternate sugar substructure. | |
| All analogs where the | —NH₂ is replaced by NR₂, where R is a | |
| H's of the terminal primary | member of the set of small alkyls, methyl, | |
| amine —H₂ are substituted by | ethyl, n-propyl, and so forth. | |
| two identical groups | | |
| All analogs where the | —N(CH₃)₂ is by NR₂, where R is H or a | |
| methyl groups of the terminal | member of the set of small alkyls, ethyl, n- | |
| tertiary amine —N(CH₃)₂ are | propyl, and so forth. | |
| substituted by two identical | | |
| groups | | |
| All analogs where the | —NR₂ is replaced by NRR′, where R | |
| substructures attached to the | and R′ are dissimilar members of the set | |
| terminal amine nitrogen are | containing H and the small alkyls, ethyl, | |
| dissimilar | n-propyl, and so forth. | |
| All analogs of psilocybin | —NR₂H+ is replaced by —NRR′R″+, | |
| that are betaines | where R, R′ and R′″ are members of the set | |
| | of small alkyls, ethyl, n-propyl, and so forth, | |
| | and where (for clarity) R, R′ and R″ may be | |
| | the same or dissimilar. | |
| All analogs where a | ROH is replaced by RSH, where R is | |
| phenolic (—OH) may be | the indole substructure. | |
| replaced by a sulfhydryl (—SH) | | |
| All metal | —OH on the phenyl part of the indole | |
| thiophenolates | structure is replaced by —SH, and the —SH is | |
| | then reacted with metal ion, displacing the | |
| | hydrogen. | |
| Sulfate analog to | [3-(2-Dimethylaminoethyl)-1H-indol-4- | |
| psilocybin (if feasible) | yl] hydrogen sulfate | |
| Isotope Modified | | |
| All of the above | Deuterated versions of the above | |
| | molecules | |

In certain embodiments, the psychedelic is a 5-HT_{2A} agonists. Exemplary 5-HT_{2A} agonists include but are not limited to (R)-2,5-dimethoxy-4-iodoamphetamine [(R)-DOI] and 2,5-dimethoxyphenethylamine (2C—H).

In certain embodiments, the psychedelic is a selective agonist of the δ-opioid receptor. A non-limiting example of a selective agonist of the δ-opioid receptor is 4-{(R)-(3-aminophenyl)[4(4-fluorobenzyl)-piperazin-1-yl]methyl}-N,N-diethylbenzamide (AZD2327) and -(alpha-(4-Allyl-2,5-dimethyl-1-piperazinyl)-3-methoxybenzyl)-N,N-diethylbenzamide.

In certain embodiments, the psychedelic is lysergic acid diethylamide (LSD).

A number of psychedelics are present in plants and fungi. For example, psilocybin is present in in in mushrooms from the following genera: Agrocybe, Amanita, Conocybe, Galerina, Gymnopilus, Hypholoma, Inocybe, Panaeolus, Psilocybe, Pholiotina, Pluteus, and Weraroa. Exemplary Psilocybe include P. cubensis and P. subcubensis. P. semilanceata. Accordingly, the psychedelic for use in the methods of the present invention may be in the form of natural products or extracts from natural products. Methods of extracting psilocybin from mushrooms or producing psilocybin are known in the art. See U.S. Pat. No. 3,183,172 describing obtaining psilocybin and psilocin from fungal material and U.S. Pat. No. 10,519,175 directed to preparations of psylocybin and polymorphs of psylocybin.

In certain embodiments, the subject may be a human or animal. Optionally the animal is a companion animal such as a cat or dog.

### EXAMPLES

### Example 1: Escitalopram Induces ABCF1 in a Macrophage Cell Line

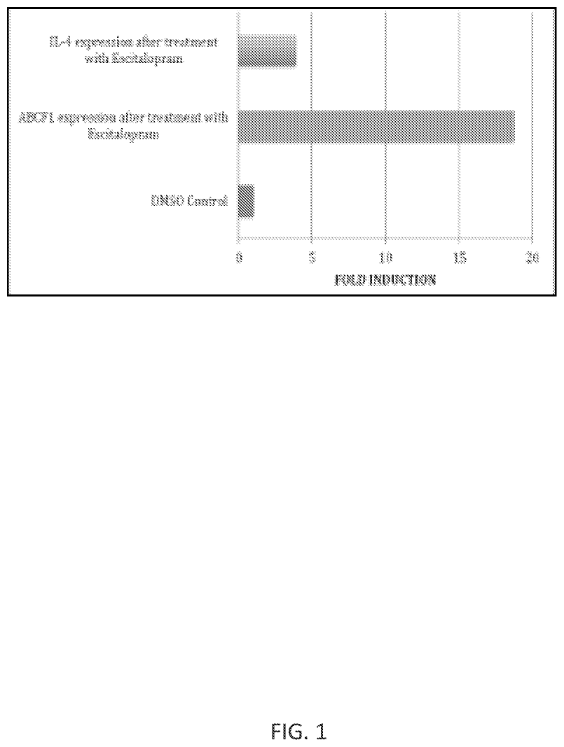

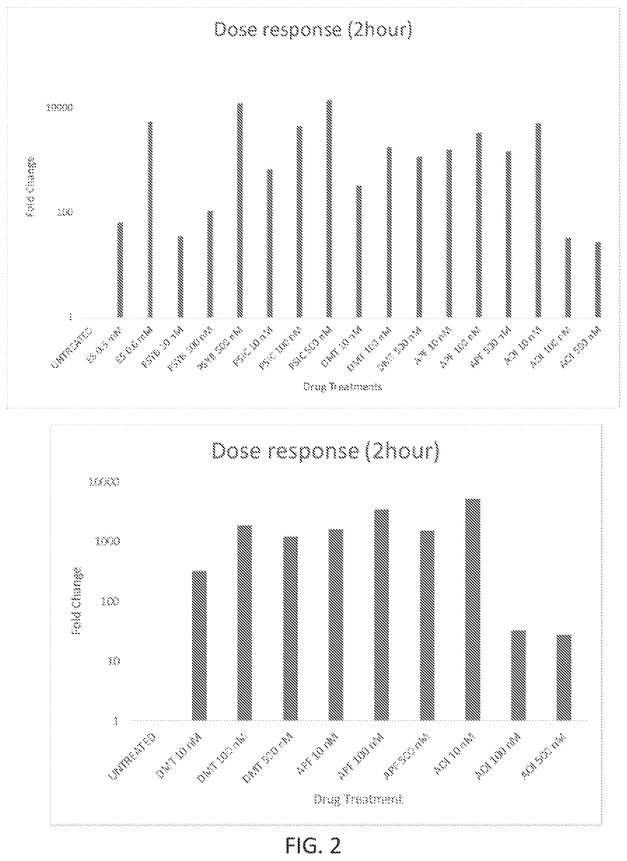

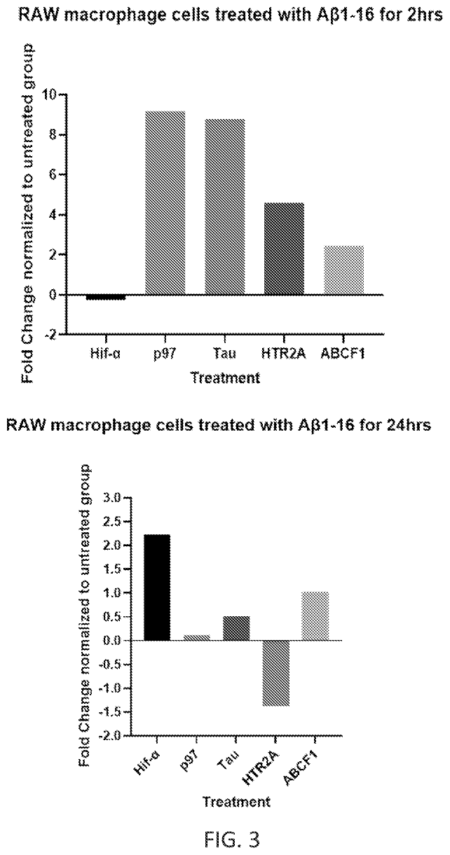

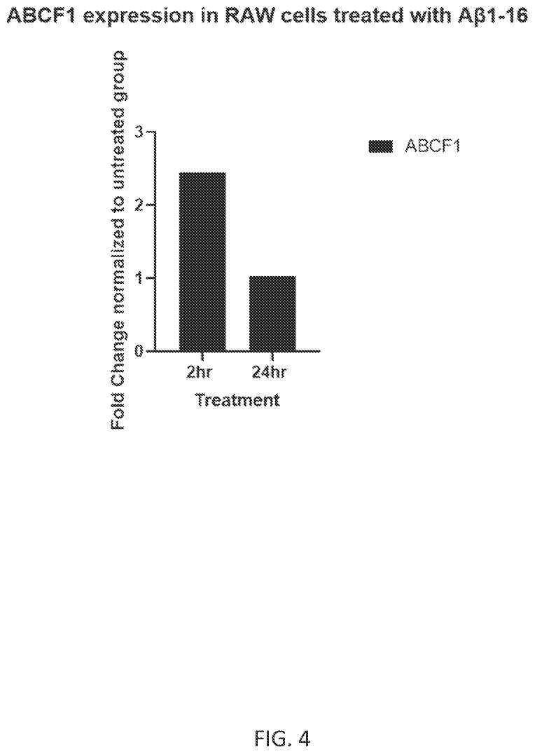

RAW macrophages were plated at 1×10⁵ cells/well and cultured for 2 days. The cells were incubated with 0.3 mM Escitalopram for 1 hour, and then harvested for total RNA, which was extracted for real time RT-PCR specific for ABCF1 and IL-4. CT values were normalized with CT value for the housekeeping gene from the DMSO control. The difference in the expression after drug treatment is consistent with polarization towards an M2-like phenotype (data were consistent in 3 separate experiments). See FIG. 1.

### Example 2: The Effect of Psylocibin, Psylocin and their Analogs on ABCF1 Transcription in a Macrophage Cell Line

### Preparation of Cells:

1. Macrophage cell line RAW264.7 (ATCC) were grown to 80% confluency in growth media (DMEM+10% FBS+glutamine).
2. Dilutions of the drugs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole.
3. Untreated cells were used as negative control and Escitalopram at 0.3 mM was used as a positive control to activate ABCF1 expression for all the experiments.
   Analysis by qPCR:

### Primers Used:

GAPDH FP:
(SEQ ID NO: 1)
TGGATTTGGACGCATTGGTC
GAPDH RP:
(SEQ ID NO: 2)
TTTGCACTGGTACGTGTTGAT
ABCF1 FP:
(SEQ ID NO: 3)
AGAAAGCCCGAGTTGTGTTTG
ABCF1 RP:
SEQ ID NO: 4)
GCCCCCTTGTAGTCGTTGATG

1. 2 hours post treatment with drugs, the reaction was stopped by removing the media with the drug. Cells were then collected and RNA was isolated from these.
2. After checking the quality of the RNA, cDNA was generated and qPCR was run with ABCF1 primers as the target gene and GAPDH as the house keeping gene.
3. Normalized against the expression level of GAPDH, the fold change expression level of ABCF1 was calculated and tabulated for all treatment conditions.

The results as set forth in FIG. 2 show psylocibin, psylocin and their analogs upregulate ABCF1 transcription.

### Example 3: Expression Levels of Markers Involved in Alzheimer's Disease Pathology in Murine Macrophage Cell Line, RAW264.7 Cells, Post Treatment with Soluble Amyloid Beta (Aβ 1-16)

### Preparation of Cells:

1. Macrophage cell line RAW264.7 (ATCC) were grown to 80% confluency in growth media DMEM+10% FBS+glutamine)
2. Confluent cells were treated with 10 uM of soluble amyloid beta (Aβ 1-16) for 2 hours and for 24 hours. Post treatment the cells were washed with PBS and cells were collected for RNA isolation followed by qRT-PCR to assess for expression levels of proteins involved in AD pathology; Hypoxia-inducible factor 1-alpha (Hif1-α), Melanotransferrin (p97), Tau protein (tau), 5-Hydroxytryptamine Receptor 2A (HTR2A), ATP Binding Cassette Subfamily F Member 1 (ABCF1)
   Analysis by qPCR:

### Primers Used

ABCF1 FP:
(SEQ ID NO: 3)
AGAAAGCCCGAGTTGTGTTTG
ABCF1 RP:
(SEQ ID NO: 4)
GCCCCCTTGTAGTCGTTGATG
App: fwd;
(SEQ ID NO: 5)
TCCGAGAGGTGTGCTCTGAA
App Rvs:
(SEQ ID NO: 6)
CCACATCCGCCGTAAAAGAATG
Tau: Fwd:
(SEQ ID NO: 7)
GAATGTCAGGTCGAAGATTGGC
Tau Rvs:
(SEQ ID NO: 8)
TGGACTGGACGTTGCTAAGAT
P97; fwd;
(SEQ ID NO: 9)
CTGAGCGTGACTTTTTGGCTA
P97 Rvs:
(SEQ ID NO: 10)
CACAGTGGTCAGCGGAGTT
Htr2A: fwd:
(SEQ ID NO: 11)
TAATGCAATTAGGTGACGACTCG
Htr2A: Rvs:
(SEQ ID NO: 12)
GCAGGAGAGGTTGGTTCTGTTT
Hif-a; fwd:
(SEQ ID NO: 13)
ACCTTCATCGGAAACTCCAAAG
Hif-a Rvs:
(SEQ ID NO: 14)
ACTGTTAGGCTCAGGTGAACT

1. Post treatment with amyloid beta at different time points, the reaction was stopped by removing the media with the drug. Cells were then collected and RNA was isolated from these
2. After checking the quality of the RNA, cDNA was generated and qPCR was run with different target primers and GAPDH as the house keeping gene
3. Normalized against the expression level of GAPDH, and further normalized against the expression level of untreated cells, the fold change expression level of different target genes was calculated and tabulated for all conditions.

### Results:

qRT-PCR shows that RAW murine macrophage cell line treated with Aβ 1-16 for 2 hours results in an upregulation of target proteins involved in inflammation and AD pathology; p97, Tau, HTR2A and ABCF1 compared to untreated cells.
qRT-PCR shows that RAW murine macrophage cell line treated with Aβ 1-16 for 24 hours results in an upregulation of Hif-α, Tau and ABCF1 and downregulation of HTR2A compared to untreated cells.
qRT-PCR shows an upregulation of ABCF1 more than two folds post a 2-hour treatment of RAW murine macrophage cells with Aβ 1-16 when compared to untreated cells. After a 24-hour treatment the expression level of ABCF1 falls by half compared to the 2 hour timepoint.

### Example 4: Expression Levels of Markers Involved in Alzheimer's Disease Pathology in Murine Macrophage Cell Line, RAW264.7 Cells, Microglia Model, Post Treatment with Soluble Amyloid Beta (Aβ 1-16) or Psychedelics

### Preparation of Cells:

1. Macrophage cell line RAW264.7 (ATCC) were grown to 80% confluency in growth media (DMEM+10% FBS+glutamine).
2. Confluent cells were treated with 10 uM of soluble amyloid beta (Aβ 1-16) for 2 hours or psychedelics at the prescribed concentrations at 2 hours post-stimulation or 24 or after restimualtion at 26 hours. Post treatment the cells were washed with PBS and cells were collected for RNA isolation followed by qRT-PCR to assess for expression levels of proteins involved in AD pathology; Hypoxia-inducible factor 1-alpha (Hif1-α), ATP Binding Cassette Subfamily F Member 1 (ABCF1).
   Analysis by qPCR:
3. Post treatment with amyloid beta at different time points, the reaction was stopped by removing the media with the drug. Cells were then collected and RNA was isolated from these cells.
4. After checking the quality of the RNA, cDNA was generated and qPCR was run with different target primers and GAPDH as the house keeping gene
5. Normalized against the expression level of GAPDH, and further normalized against the expression level of untreated cells, the fold change expression level of different target genes was calculated and tabulated for all conditions.

### Results:

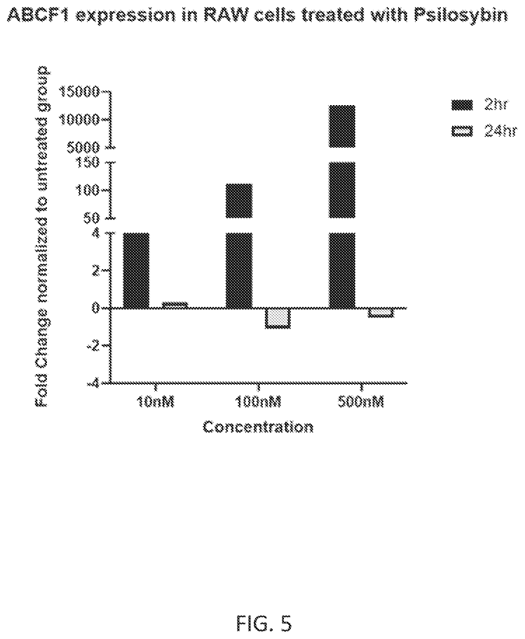

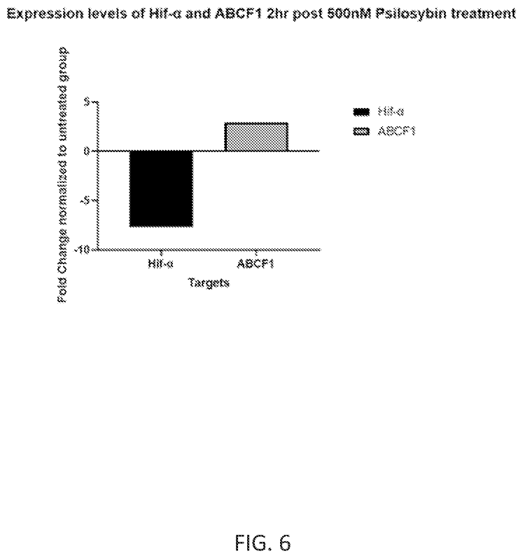

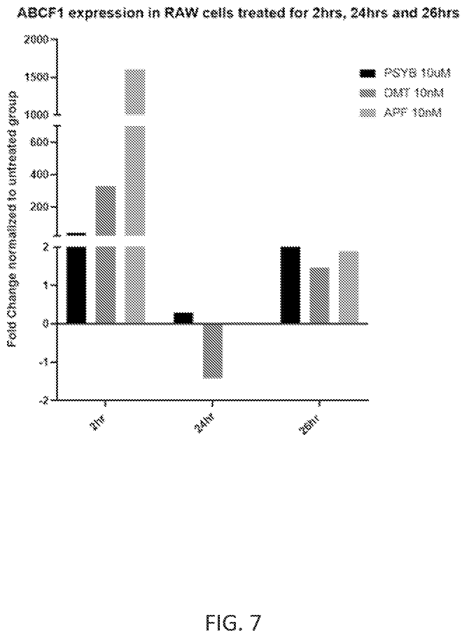

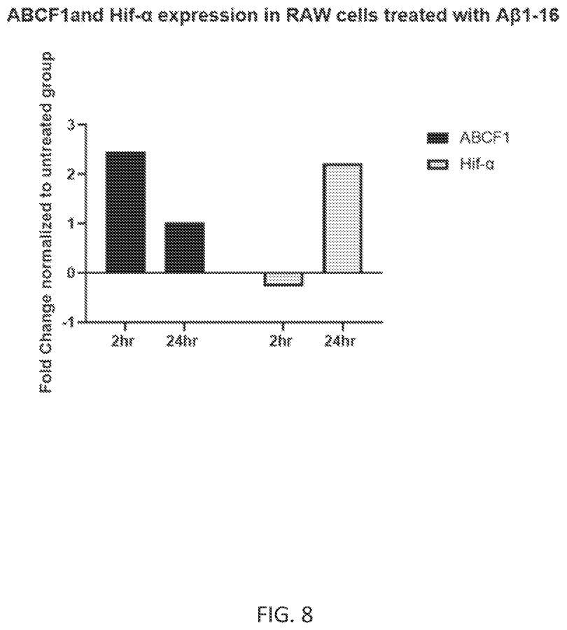

Target markers that are implicated in AD pathology and inflammation shown here to be modulated by treatment of RAW cells with psychedelics
Expression of ABCF1 is shown in FIG. 5 to be modulated by treatment of RAW cells with psychedelics for 2 or 24 hours.
Expression of Hifα and ABCF1 are shown in FIG. 6 to be modulated by treatment of RAW cells with psychedelics for 2 hours.
FIG. 7 shows the expression of ABCF1 is modulated by treatment of RAW cells with psychedelics for 2 hours incubation, 24 and after 2 hours of re-incubation the same cells for 2 hours 24-26 hours.
qRT-PCR shows that RAW murine macrophage cell line treated with Aβ 1-16 for 2 hours results in an upregulation of target proteins involved in inflammation and AD pathology; HIF-alpha and ABCF1 compared to untreated cells (FIG. 8)

### Example 5: Down Regulation of APP and Tau by Psychedelics

Amyloid precursor protein (APP) and Tau are involved in the pathogenesis of Alzheimer's disease. In particular, Alzheimer's disease is characterized by amyloid plaques formed by extracellular aggregates of amyloid-beta (Aβ) peptides which are fragments of APP and neurofibrillary tangles which consist of intracellular aggregates of phosphorylated tau (p-tau) protein.

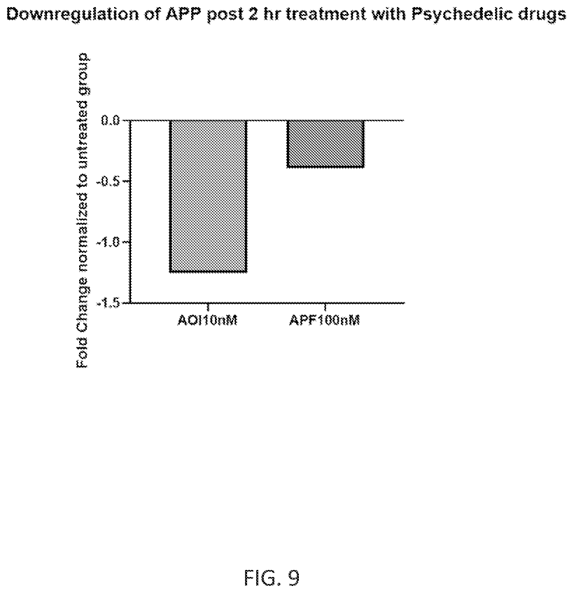

FIG. 9 illustrates down regulation of amyloid precursor protein (APP) in RAW cells post 2 hours treatment with psychedelic drugs (APF=O-Acetyl Psilocin Fumerate, and AOI=4-acetoxyindole).

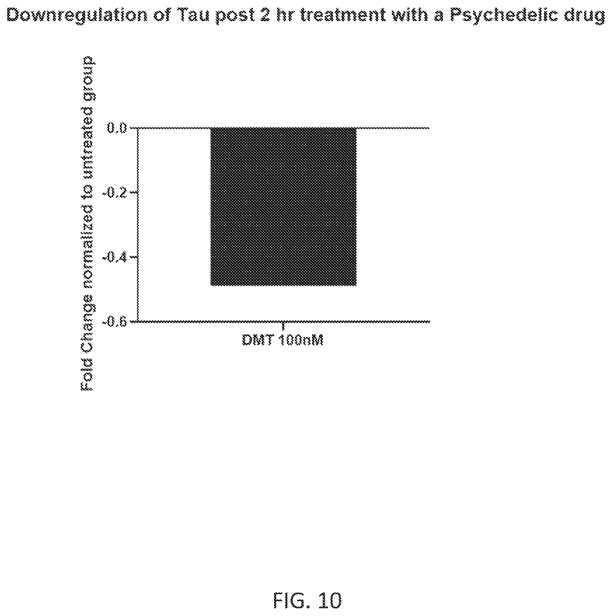

FIG. 10 illustrates down regulation of Tau in RAW cells post 2 hours treatment with psychedelic drugs (DMT=4-Acetoxy-N, N-dimthyltryptamine).

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of inhibiting neuroinflammation by upregulating the expression and/or activity of ABCF1.

2. The method of claim 1, wherein inhibiting neuroinflammation treats, delays and/or prevents dementia.

3. The method of claim 1, wherein said upregulating the expression and/or activity of ABCF1 is by administration of one or more psychedelics.

4. The method of claim 3, wherein the one or more psychedelics are selected from the group consisting of psilocybin, psilocin, 4-Acetoxy-N, N-dimthyltryptamine; O-Acetyl Psilocin Fumerate, and 4-acetoxyindole.

5. A method of inhibiting neuroinflammation and thereby treating, delaying and/or preventing dementia by administering one or more psychedelics alone or in combination with other therapeutics.

6. The method of claim 5, wherein said one or more psychedelics upregulate the expression and/or activity of ABCF1.

7. The method of claim 5, wherein said one or more psychedelics are selected from psilocybin, psilocybin derivatives and psilocybin like compounds.

8. A method of treating, delaying and/or preventing dementia by administering one or more psychedelics alone or in combination with other therapeutics.

9. The method of claim 8, wherein the one or more psychedelics inhibit neuroinflammation, inhibit cerebral angiogenesis, promote neuroplasticity, down regulate APP and/or Tau, restore blood brain barrier or a combination thereof and thereby treat, delay and/or prevent dementia.

10. The method of claim 8, wherein said one or more psychedelics are selected from psilocybin, psilocybin derivatives and psilocybin like compounds.

11. The method of claim 1, wherein the dementia is Alzheimer's disease.

12. The method of claim 1, wherein the dementia is Downs syndrome dementia, vascular dementia, frontal temporal or Picks disease.


SEQUENCE LISTING (14 sequences)

SEQ ID NO: 1 - DNA, 20 bp, Artificial Sequence
  GAPDH FP
tggatttggacgcattggtc

SEQ ID NO: 2 - DNA, 21 bp, Artificial Sequence
  GAPDH RP
tttgcactggtacgtgttgat

SEQ ID NO: 3 - DNA, 21 bp, Artificial Sequence
  ABCF1 FP
agaaagcccgagttgtgtttg

SEQ ID NO: 4 - DNA, 21 bp, Artificial Sequence
  ABCF1 RP
gcccccttgtagtcgttgatg

SEQ ID NO: 5 - DNA, 20 bp, Artificial Sequence
  App fwd
tccgagaggtgtgctctgaa

SEQ ID NO: 6 - DNA, 22 bp, Artificial Sequence
  App  Rvs
ccacatccgccgtaaaagaatg

SEQ ID NO: 7 - DNA, 22 bp, Artificial Sequence
  Tau Fwd
gaatgtcaggtcgaagattggc

SEQ ID NO: 8 - DNA, 21 bp, Artificial Sequence
  Tau  Rvs
tggactggacgttgctaagat

SEQ ID NO: 9 - DNA, 21 bp, Artificial Sequence
  P97 fwd
ctgagcgtgactttttggcta

SEQ ID NO: 10 - DNA, 19 bp, Artificial Sequence
  P97  Rvs
cacagtggtcagcggagtt

SEQ ID NO: 11 - DNA, 23 bp, Artificial Sequence
  Htr2A fwd
taatgcaattaggtgacgactcg

SEQ ID NO: 12 - DNA, 22 bp, Artificial Sequence
  Htr2A Rvs
gcaggagaggttggttctgttt

SEQ ID NO: 13 - DNA, 22 bp, Artificial Sequence
  Hif-a fwd
accttcatcggaaactccaaag

SEQ ID NO: 14 - DNA, 21 bp, Artificial Sequence
  Hif-a  Rvs
actgttaggctcaggtgaact